(19) United States
(12) Patent Application Publication
(10) Pub. No.: US 2026/0308703 A1
(43) Pub. Date: Oct. 8, 2026

(54) CRISPR-CAS-BASED NOVEL GUIDE RNA CAPABLE OF SPECIFIC DETECTION OF NOROVIRUS GII.4 AND APPLICATION THEREOF

(71) Applicant: PULMUONE CO., LTD., Eumseong-gun,Chungcheongbuk-do (KR)

(72) Inventors: Sang Yong LEE, Cheongju-si, Chungcheongbuk-do (KR); Young Min PARK, Cheongju-si, Chungcheongbuk-do (KR); Sang Gu KIM, Cheongju-si, Chungcheongbuk-do (KR); Sang Yoon LEE, Cheongju-si, Chungcheongbuk-do (KR); Hyo Yul LEE, Cheongju-si, Chungcheongbuk-do (KR)

(21) Appl. No.: 19/482,496

(22) PCT Filed: Aug. 17, 2023
(86) PCT No.: PCT/KR2023/012181

§ 371 Date: Nov. 7, 2025

(30) Foreign Application Priority Data
May 9, 2023 (KR) 10-2023-0059731

(51) Int. Cl. C12Q 1/70; C12N 9/22; C12Q 1/44; C12Q 1/6816; C12Q 1/6844; C12Q 1/6876

(52) U.S. Cl. CPC C12Q 1/701; C12N 9/226; C12Q 1/44; C12Q 1/6816; C12Q 1/6844; C12Q 1/6876; G01N 2800/42

(57) ABSTRACT

The present invention relates to a CRISPR-Cas-based novel guide RNA capable of specific detection of norovirus GII.4 and a method for, by using same, the detection of norovirus GII.4 and the diagnosis of infection thereby. Using the guide RNA provided by the present invention, norovirus GII.4 in specimens can be rapidly detected with very high sensitivity and specificity.

### TECHNICAL FIELD

The present application claims priority to Korean Patent Application No. 10-2023-0059731, filed on May 9, 2023, and the entire disclosure of the aforementioned application is incorporated herein by reference.

The present invention relates to a novel guide RNA specific for norovirus GII.4 that enables CRISPR-Cas-based detection of norovirus GII.4, and to a method for detecting norovirus GII.4 and diagnosing infections thereof using the same.

### BACKGROUND ART

Norovirus is known worldwide as the causative agent of non-bacterial acute gastroenteritis and is a type of enteric virus that causes infectious gastroenteritis in humans when contaminated food or water is consumed. Norovirus belongs to the family Caliciviridae and has a single-stranded RNA genome of approximately 7.5 kb in size. It consists of three open reading frames (ORFs). Norovirus was first discovered in the diarrhea samples of mass food poisoning patients at an elementary school in Norwalk, Ohio, USA, in 1968, and at that time, it was referred to as Norwalk Viruses. In August 2002, the International Committee on Taxonomy of Viruses (ICTV) unified the name to Norovirus. Currently, among the five genotypes of Norovirus classified by genotype (GI, GII, GIII, GIV, GV), the genotypes that cause infections in humans are the GI, GII, and GIV groups. Among these, genotype GII.4 is involved in most Norovirus food poisoning cases worldwide, and in Korea, GII.4 is followed by GII.3, which has been reported with a high detection rate.

When infected with norovirus, the main symptoms include nausea, vomiting, diarrhea, and abdominal pain. In most cases, the symptoms are mild and resolve naturally within 1 to 2 days. The incubation period is 24 to 48 hours, and the virus from the feces or vomit of an infected person can contaminate food or water, or objects and food contacted by the infected person can become contaminated. The virus can enter the mouth by eating, drinking, or coming into contact with these contaminated items. Even a small amount of the virus can easily cause infection, and it spreads easily. Infectivity is highest during the symptomatic period and can persist from 3 days to up to 2 weeks after recovery (Korea Disease Control and Prevention Agency Health Information).

Commercially available enzyme immunoassay (EIA) diagnostic kits for detecting norovirus antigens have been developed. However, the enzyme immunoassay method has the disadvantage of varying sensitivity depending on the norovirus strain. For this reason, it can be used as an auxiliary means for identifying the cause of norovirus outbreaks but has been reported to have technical limitations for use as a standard protocol for diagnosis. Generally, reverse transcription polymerase chain reaction (RT-PCR) and real-time reverse transcription polymerase chain reaction (Real-Time RT-PCR) methods, which directly amplify and detect viral genes from the feces of patients, are widely used for detection and diagnosis. However, these methods have the disadvantage of requiring high costs for primer or probe production because it is difficult to achieve high sensitivity detection with a single primer pair.

Recently, CRISPR-Cas-based DNA detection platforms, including SHERLOCK (Cas13), DETECTR (Cas12a), and Cas14-DETECTR, which have high detection sensitivity and specificity, have been developed. However, to date, no related technology has been developed to detect Norovirus GII.4 with very high specificity.

Therefore, to improve the accuracy and sensitivity of detecting norovirus GII.4 and diagnosing its infection, it is necessary to develop a novel guide RNA used in CRISPR-Cas-based gene detection methods and methods utilizing the same.

### Problem to be Solved

Accordingly, the present inventors have completed the present invention by developing a novel guide RNA capable of specifically detecting norovirus GII.4 in conjunction with CRISPR-Cas proteins and evaluating its effectiveness.

Therefore, an object of the present invention is to provide a guide RNA specific for norovirus GII.4 comprising the nucleotide sequence of SEQ ID NO: 1.

Another object of the present invention is to provide a composition and a kit for detecting norovirus GII.4 comprising the guide RNA.

In addition, another object of the present invention is to provide a composition and a kit for detecting norovirus GII.4 consisting of the guide RNA.

Furthermore, another object of the present invention is to provide a composition and a kit for detecting norovirus GII.4 consisting essentially of the guide RNA.

Another object of the present invention is to provide a method for detecting norovirus GII.4 comprising:
(a) amplifying nucleic acid obtained from a sample;
(b) adding the guide RNA, an endonuclease, and a reporter gene to the amplification product and reacting; and
(c) detecting a signal of the reporter gene.

Another object of the present invention is to provide a composition for diagnosing food poisoning comprising the guide RNA.

In addition, another object of the present invention is to provide a composition for diagnosing food poisoning consisting of the guide RNA.

Furthermore, another object of the present invention is to provide a composition for diagnosing food poisoning consisting essentially of the guide RNA.

Another object of the present invention is to provide the use of the guide RNA for preparing a composition for diagnosing food poisoning.

Another object of the present invention is to provide a method for diagnosing food poisoning comprising:
(a) amplifying nucleic acid obtained from a sample;
(b) adding the guide RNA, an endonuclease, and a reporter gene to the amplification product and reacting;
(c) detecting a signal of the reporter gene; and
(d) diagnosing as food poisoning when the signal of the reporter gene is detected in step c).

However, the problems of the present invention as described above are exemplary, and the scope of the present invention is not limited thereby. In addition, other objects and advantages of the present invention will become more apparent from the following detailed description of the invention, claims, and drawings.

### Means for Solving the Problem

To achieve the aforementioned objectives of the present invention, the present invention provides a guide RNA specific for norovirus GII.4 comprising the nucleotide sequence of SEQ ID NO: 1.

To achieve another objective of the present invention, the present invention provides a composition and a kit for detecting norovirus GII.4 comprising the guide RNA.

Additionally, to achieve another objective of the present invention, the present invention provides a composition and a kit for detecting norovirus GII.4 consisting of the guide RNA.

Furthermore, to achieve another objective of the present invention, the present invention provides a composition and a kit for detecting norovirus GII.4 consisting essentially of the guide RNA.

To achieve another objective of the present invention, the present invention provides a method for detecting norovirus GII.4 comprising:
(a) amplifying nucleic acid obtained from a sample;
(b) adding the guide RNA, an endonuclease, and a reporter gene to the amplification product and reacting; and
(c) detecting a signal of the reporter gene.

To achieve another objective of the present invention, the present invention provides a composition for diagnosing food poisoning comprising the guide RNA.

Additionally, to achieve another objective of the present invention, the present invention provides a composition for diagnosing food poisoning consisting of the guide RNA.

Furthermore, to achieve another objective of the present invention, the present invention provides a composition for diagnosing food poisoning consisting essentially of the guide RNA.

To achieve another objective of the present invention, the present invention provides the use of the guide RNA for preparing a composition for diagnosing food poisoning.

To achieve another objective of the present invention, the present invention provides a method for diagnosing food poisoning comprising:
(a) amplifying nucleic acid obtained from a sample;
(b) adding the guide RNA, an endonuclease, and a reporter gene to the amplification product and reacting;
(c) detecting a signal of the reporter gene; and
(d) diagnosing as food poisoning when the signal of the reporter gene is detected in step c.

Unless otherwise defined, all technical and scientific terms used herein have the same meaning as commonly understood by one of ordinary skill in the art.

Hereinafter, the present invention will be described in detail.

The present invention provides a guide RNA specific for norovirus GII.4 comprising the nucleotide sequence of SEQ ID NO: 1.

In the present invention, the guide RNA may be crRNA or gRNA. crRNA is referred to as CRISPR RNA. Additionally, gRNA refers to guide RNA. crRNA and gRNA may be single-stranded RNA. Furthermore, crRNA may bind to tracrRNA to activate CRISPR-associated proteins, and crRNA may be used in a form bound to tracrRNA. In this case, crRNA may have a sequence complementary to a gene sequence specifically present in norovirus GII.4. Additionally, gRNA may also bind to a gene sequence specifically present in norovirus GII.4 to activate CRISPR-associated proteins. The crRNA or gRNA may be RNA consisting of 15 to 40 nucleotides. In one specific example, the crRNA or gRNA may consist of 24 nucleotides. Furthermore, the crRNA or gRNA may include additional sequences (e.g., tracrRNA, scoutRNA) at the 3′ end to activate CRISPR-associated proteins such as Cas12, Cas13, or Cas14. The guide RNA may include a polynucleotide complementary to a nucleotide sequence of 2 to 24 consecutive nucleotides (e.g., approximately 20 nt, hereinafter referred to as “nt”) in the 5′ or 3′ direction of the PAM in the target nucleic acid. The length of the guide RNA may be 10 nt to 100 nt, 10 nt to 90 nt, 10 nt to 80 nt, 10 nt to 70 nt, 10 nt to 60 nt, 10 nt to 50 nt, 15 nt to 50 nt, 20 nt to 50 nt, 25 nt to 50 nt, 30 nt to 50 nt, 35 nt to 50 nt, 40 nt to 50 nt, or 45 nt to 50 nt. In one embodiment, the gRNA may be characterized by comprising the nucleotide sequence of SEQ ID NO: 1.

The present invention also provides a composition for detecting norovirus GII.4 comprising the guide RNA.

The composition according to the present invention may further comprise additional components for specifically detecting norovirus GII.4 using the guide RNA, specifically an endonuclease and a reporter gene, and may further comprise a primer pair including a sequence complementary to the norovirus GII.4 gene to amplify it.

In the present invention, the term “primer” refers to a short nucleic acid sequence with a free 3′ hydroxyl group that can form base pairs with a complementary template of norovirus nucleic acid and functions as a starting point for template strand replication. The composition according to the present invention may include any primer pair that includes a sequence complementary to the gene of norovirus GII.4 and is capable of amplifying it. Preferably, the primer pair may specifically amplify the VP1 capsid region of the ORF2 gene of norovirus GII.4.

In one embodiment of the present invention, the primer pair may include a forward primer comprising the nucleotide sequence selected from the group consisting of SEQ ID NOs: 2 to 6 and a reverse primer comprising the nucleotide sequence of SEQ ID NO: 7, preferably a forward primer comprising the nucleotide sequence of SEQ ID NO: 5 and a reverse primer comprising the nucleotide sequence of SEQ ID NO: 7, and most preferably a forward primer consisting of the nucleotide sequence of SEQ ID NO: 5 and a reverse primer consisting of the nucleotide sequence of SEQ ID NO: 7. The forward primer selected from the group consisting of SEQ ID NOs: 2 to 6 and the reverse primer of SEQ ID NO: 7 specifically amplify the VP1 capsid region of the ORF2 gene of norovirus GII.4, and norovirus GII.4 can be detected through the analysis of the amplification product.

The amplification may be performed by any method used in the art to amplify a target (e.g., gene), for example, by recombinase polymerase amplification (RPA), but is not limited thereto.

The amplification product may, for example, be an amplicon, but is not limited thereto.

In the present specification, the term “recombinase polymerase amplification (RPA)” refers to a technique for detecting DNA and RNA amplification, which, unlike conventional PCR, uses DNA-binding proteins and recombinase to rapidly and accurately amplify target sequences. RPA is similar to conventional PCR but can amplify specific genes under isothermal conditions without temperature changes, thereby reducing reaction time, eliminating the need for specialized equipment, and enabling cost-effective, portable, and on-site testing. Recombinase polymerase amplification (RPA) uses bacteriophage T4 recombinase to dissociate double-stranded DNA and simultaneously amplify specific DNA using DNA polymerase and specific primers. Like PCR, it uses a target template and a pair of primers to amplify specific DNA sequences, but unlike PCR, it induces amplification reactions under isothermal conditions in the range of 37° C. to 42° C. RPA has been developed to rapidly produce amplification products, which, along with the advantages of isothermal conditions, is widely recognized and applied to various methods for detecting specific pathogens through specific gene amplification.

The length of RPA amplification products is recommended not to exceed 500 nt (nucleotides) and generally ranges between 100 and 250 nt. Unlike PCR, the primer length for RPA is recommended to be at least 30 nucleotides, with a typical length of 32 to 35 nt. Additionally, unlike PCR, RPA primers are not sensitive to the GC content (GC %) and Tm (melting temperature) value, so RPA primers should converge to GC % (20-70%) and Tm (50-100° C.) (Analyst, 2019, 144, 31).

In the present invention, the endonuclease may be a CRISPR-associated protein. The term “CRISPR-associated protein” used in the present invention refers to an enzyme that can recognize and cleave nucleic acids such as DNA or RNA, whether double-stranded or single-stranded (dsDNA/RNA and ssDNA/RNA). Specifically, these enzymes can recognize and cleave double-stranded or single-stranded nucleic acids bound to crRNA or gRNA.

A specific example of the endonuclease in the present invention may be one in which the endonuclease function is activated upon recognizing that the gRNA is bound to the target site. Additionally, as the endonuclease function is activated, it may have exonuclease activity capable of nonspecifically cleaving double-stranded and/or single-stranded DNA and/or RNA. Furthermore, CRISPR-associated proteins such as Cas12, Cas13, or Cas14 may exhibit nonspecific exonuclease activity once activated. They may exhibit activity to cleave target nucleic acids bound to guide RNA (cis-cleavage) or nonspecifically cleave external arbitrary nucleic acids, specifically reporter nucleic acids (DNA and/or RNA) (trans-cleavage).

Therefore, in one specific example of the composition of the present invention, norovirus GII.4 can be detected by the fluorescence emission of a fluorescent substance bound to the reporter, which is cleaved by a nonspecific nuclease activated by the binding of crRNA or gRNA to a specific target site of norovirus GII.4.

Specifically, one specific example of the CRISPR-associated protein may be Cas12, Cas13, or Cas14, and specifically, it may be any one selected from the group consisting of Cas12a, Cas12b, Cas12c, Cas12d, Cas12e, Cas12g, Cas12h, Cas12i, Cas13a, Cas13b, Cas13c, Cas13d, and Cas14.

Cas12 and Cas14 may exhibit endonuclease activity and nonspecific exonuclease activity in the presence or absence of a PAM sequence in the target nucleic acid after binding to guide RNA complementary to the target nucleic acid, and may cleave ssDNA of arbitrary sequences or dsDNA of the target nucleic acid.

Cas13 may cleave ssRNA of arbitrary sequences or ssRNA of the target nucleic acid after binding to guide RNA complementary to the target nucleic acid.

In the region of the norovirus GII.4 gene to which the guide RNA according to the present invention complementarily binds, a TTTA PAM sequence is present. Therefore, in order for endonuclease or exonuclease activity to be exerted, the present invention can be applied in combination with CRISPR-associated proteins that require PAM sequences such as (T) TTV, TTN, TN, or do not require PAM sequences for endonuclease or exonuclease activity.

In the present invention, the reporter gene refers to DNA or RNA of arbitrary sequences cleaved by the exonuclease activity of CRISPR-associated proteins, and it is preferable that the reporter gene is labeled with a detectable label. The detectable label may be bound to the terminal or internal region of the reporter gene. The detectable label may be a chemical label, enzyme label, radioactive label, fluorescent label, luminescent label, chemiluminescent label, FRET (fluorescence resonance energy transfer) label, metal label, or a combination thereof. The fluorescent label may be selected from the group consisting of FAM, TET, JOE, YAKYE, HEX, CY3, ATTO550, TAM, ROX, TxRed, CY35, LC610, LC640, ATTO647N, CY5, VIC, evergreen dye, and ATTO680, but is not limited thereto.

In one aspect of the present invention, the composition may be characterized by being used in a method for detecting norovirus GII.4 through an isothermal amplification reaction and a subsequent CRISPR gene-editing reaction.

Specifically, if a target site exists within the amplification product amplified through a primer pair capable of amplifying the norovirus GII.4 gene, preferably a primer pair capable of specifically amplifying the VP1 capsid region of the ORF2 gene of norovirus GII.4, the guide RNA binds complementarily to the target site, and the endonuclease, preferably a CRISPR-associated protein, is activated to cleave the sequence of the reporter gene. As a result, norovirus GII.4 can be detected by the labeling substance bound to the reporter gene.

The present invention also provides a kit for detecting norovirus GII.4 comprising the composition.

The kit may be a kit comprising essential elements necessary for performing the detection of norovirus GII.4, and in addition to the composition, it may include test tubes or other suitable containers, reaction buffers (with varying pH and magnesium concentrations), deoxynucleotides (dNTPs), enzymes such as Taq polymerase, DNase, RNase inhibitors, DEPC-water, and sterile water.

The present invention also provides a method for detecting norovirus GII.4 comprising:
(a) amplifying nucleic acid obtained from a sample;
(b) adding the guide RNA according to claim 1, an endonuclease, and a reporter gene to the amplification product and reacting; and
(c) detecting a signal of the reporter gene.

In the present invention, the “sample” refers to a material or organism used for testing, examination, or analysis, and its type is not particularly limited, but it may be food or a biological sample suspected of containing norovirus GII.4. The sample may be a patient requiring the therapeutic effect.

In the present invention, the biological sample is obtained by isolating from a specimen suspected of a disease and may include tissue, cells, whole blood, serum, plasma, cerebrospinal fluid, colostrum, synovial fluid, saliva, feces, cell culture supernatant, etc., but is not limited thereto.

Nucleic acid obtained from the sample can be acquired using conventional known nucleic acid acquisition methods. If the starting material is gDNA, the separation of gDNA can be performed according to conventional methods known in the art, and if the starting material is mRNA, it can be synthesized into cDNA using reverse transcriptase. For example, the RNA separation is not particularly limited as long as it is a method for separating RNA from a sample. For instance, commercially available RNA separation kits such as the RNeasy mini kit from Qiagen may be used.

In the present invention, the “amplification” may be any one selected from the group consisting of PCR, RT (reverse transcription)-PCR, isothermal amplification (RPA), and RT (reverse transcription)-RPA, preferably RPA or RT-RPA, and most preferably RT-RPA.

The “isothermal amplification” refers to a method for amplifying a target nucleic acid sequence (template) under constant reaction temperature conditions, and may be any one of recombinase polymerase amplification (RPA), loop-mediated isothermal amplification (LAMP), helicase-dependent amplification (HDA), rolling circle amplification (RCA), multiple displacement amplification (MDA), strand displacement amplification (SDA), nucleic acid sequence-based amplification (NASBA), and preferably recombinase polymerase amplification (RPA).

The detection of the amplification product may be performed through capillary electrophoresis, DNA chips, gel electrophoresis, radioactive measurement, fluorescence measurement, or phosphorescence measurement, but is not limited thereto.

As one method for detecting the amplification product, for example, capillary electrophoresis may be performed. Capillary electrophoresis may use, for example, an ABi Sequencer. Additionally, gel electrophoresis may be performed, and gel electrophoresis may use agarose gel electrophoresis or acrylamide gel electrophoresis depending on the size of the amplification product. Furthermore, the fluorescence measurement method may label the 5′-end of the primer with Cy-5 or Cy-3 to perform PCR, and the target sequence is labeled with a detectable fluorescent label, and the labeled fluorescence can be measured using a fluorescence detector. Additionally, the radioactive measurement method may add radioactive isotopes such as 32P or 35S to the PCR reaction solution during PCR to label the amplification product, and the radioactivity can be measured using radioactive measuring instruments, for example, a Geiger counter or a liquid scintillation counter. Additionally, the RPA amplification product can be obtained and separated by methods such as agarose gel electrophoresis, and the presence of DNA with a length corresponding to the DNA polymerized by the primer set used, specifically DNA with a length of 151 bp, can be confirmed to detect norovirus GII.4.

In one aspect of the present invention, when the endonuclease is Cas13, a step of transcribing the amplification product may additionally be performed.

A specific example of detecting norovirus GII.4 using the method of the present invention is as follows:
(1) Amplify the gene of norovirus GII.4 present in the sample using a primer pair. If the gene is DNA, amplify the gene using PCR, RPA, etc. If the gene is RNA, amplify the gene using RT (reverse transcription)-PCR, RT-RPA, etc.
(2) In the case of Cas13, the amplification product is transcribed (e.g., T7 transcription) to perform an additional process of converting it into RNA, whereas Cas12 and Cas14 use the amplification product of (1) as is.
(3) The guide RNA, Cas12 or Cas14, and reporter DNA are mixed with the amplification product of (1) to react, or the guide RNA, Cas13, and reporter RNA are mixed with the transcript of the amplification product of (2) to react.
(4) The signal generated by the cleavage of the reporter is detected.

The present invention also provides a composition for diagnosing food poisoning.

The present invention provides the use of the guide RNA for preparing a composition for diagnosing food poisoning.

The present invention provides a method for diagnosing food poisoning comprising:
(a) amplifying nucleic acid obtained from a sample;
(b) adding the guide RNA according to claim 1, an endonuclease, and a reporter gene to the amplification product and reacting;
(c) detecting a signal of the reporter gene; and
(d) diagnosing as food poisoning when the signal of the reporter gene is detected in step (c).

In one embodiment, the present invention provides a method for diagnosing and treating food poisoning of a sample comprising:
(i) amplifying nucleic acid obtained from a sample;
(ii) adding the guide RNA according to claim 1, an endonuclease, and a reporter gene to the amplification product and reacting;
(iii) detecting a signal of the reporter gene;
(iv) diagnosing as food poisoning when the signal of the reporter gene is detected in step (iii); and
(v) administering a therapeutic agent for treating food poisoning or performing surgery to treat the disease in the diagnosed sample.

The methods comprising steps (i) to (v) are understood in accordance with the methods comprising steps (a) to (d) described above.

Step (v) is a step of treating the disease in the sample diagnosed with the disease in step (iv) through means such as administering therapeutic agents like ciprofloxacin, azithromycin, or ceftriaxone, or performing surgery.

The term “diagnosis” as used herein includes determining whether an object currently has a specific disease or condition, or determining the prognosis of an object affected by a specific disease or condition.

The “treatment” of the present invention comprehensively refers to improving food poisoning or its symptoms, which may include curing the disease, substantially preventing it, or improving the condition, and includes alleviating, curing, or preventing one or more symptoms caused by the disease, but is not limited thereto.

The term “comprising” as used herein is used in the same sense as “including” or “characterized by” and does not exclude additional components or steps not specifically mentioned in the composition or method according to the present invention. The term “consisting of” means excluding additional elements, steps, or components not separately described. The term “consisting essentially of” means that the composition or method may include substances or steps that do not substantially affect its basic characteristics, in addition to the described substances or steps.

The composition comprising the guide RNA provided by the present invention can specifically detect norovirus GII.4, and thus, it can rapidly and accurately detect norovirus GII.4 in a biological sample to be diagnosed (e.g., feces, blood, etc.).

Therefore, it enables very rapid and accurate diagnosis of diseases caused by norovirus GII.4 infection, specifically food poisoning.

### Effect of the Invention

By using the guide RNA provided by the present invention, norovirus GII.4 in a sample can be rapidly detected with very high sensitivity and specificity.

### BRIEF DESCRIPTION OF THE DRAWINGS

FIG. 1 is a diagram showing the position and sequence within the target gene and the PAM sequence targeted by the gRNA for detecting norovirus GII.4.

FIG. 2 is a diagram showing the process and results of constructing a primer pair combination capable of amplifying the gene of norovirus GII.4 with high sensitivity.

FIG. 3 shows the experimental conditions for amplifying the gene of norovirus GII.4 using a primer pair and detecting norovirus GII.4 using the amplification product, gRNA, CRISPR-Cas12a (Cpf1), and a reporter gene.

FIG. 4 shows the results of confirming the detection sensitivity by amplifying the gene of norovirus GII.4 using a primer pair and detecting norovirus GII.4 using the amplification product, gRNA, CRISPR-Cas12a (Cpf1), and a reporter gene.

FIG. 5 shows the results of confirming the detection specificity by detecting various viruses using the gRNA according to the present invention (MBC111-R: norovirus GII.3, NCCP76067: norovirus GII.4).

### DETAILED DESCRIPTION OF THE INVENTION

Hereinafter, preferred embodiments are presented to aid in understanding the present invention. However, the following embodiments are provided merely to facilitate understanding of the present invention, and the content of the present invention is not limited thereto.

### Example 1: Selection of gRNA for Detecting Norovirus GII.4

The core of research in the diagnostic field using CRISPR gene-editing technology lies in the applied Cas protein and gRNA. In this study, cpf1 (Cas12a), which has activity on dsDNA, was selected, and this protein is known to require a T-rich PAM (TTTV) within the target.

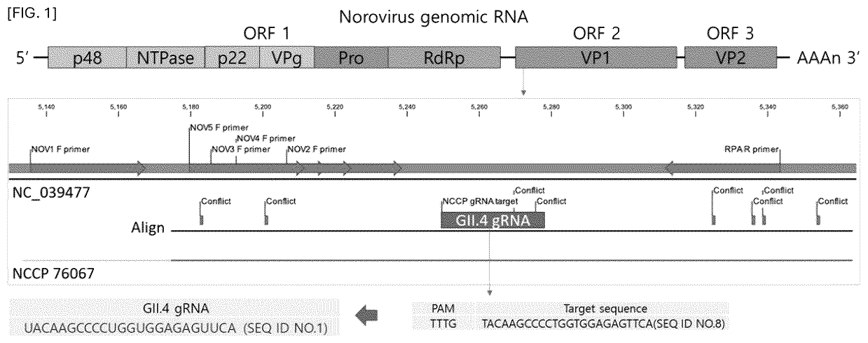

A gRNA specific to GII.4 was selected by targeting the VP1 capsid region of the Norovirus GII ORF2 gene and aligning each reference genome of the GII.4 genotype (GII.4: NC_039477) with the nucleotide sequences of standard pathogens (FIG. 1).

### Example 2: RPA Primer Design

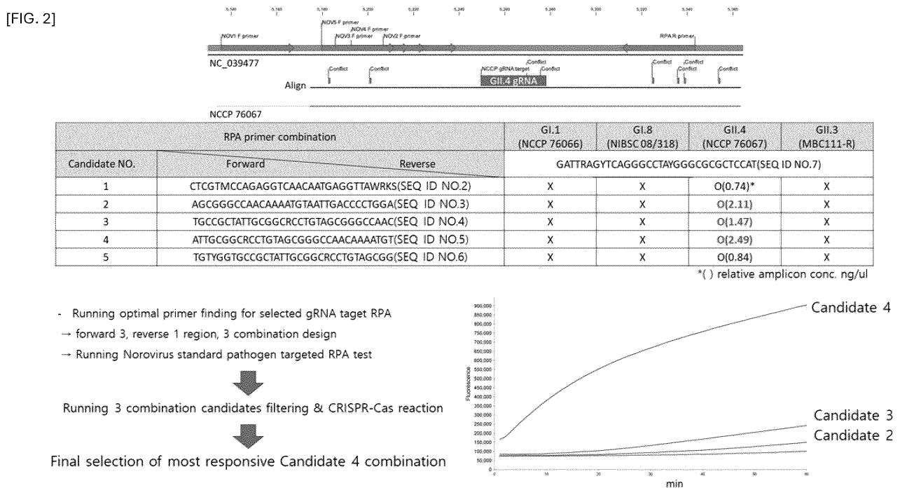

For the selected gRNA, to design the optimal RPA primer, five forward and one reverse regions, were selected, forming five combinations. RPA primer validation was performed targeting Norovirus GII.4 (NCCP 76067), and the primer pair with the best reaction was used in subsequent experiments (FIG. 2).

### Example 3: Detection Experiment of Norovirus GII.4 Using gRNA, Primer Pair, and CRISPR-Cas12a

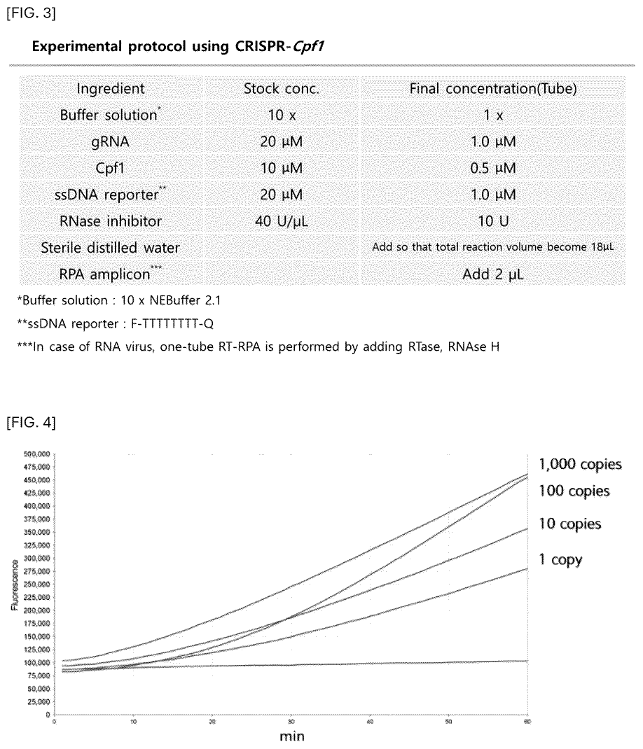

After amplifying the Norovirus GII.4 gene using the finally selected primer pair, a reaction applying gRNA and CRISPR-Cas12a was conducted, and the detailed reaction conditions are shown in FIG. 3. The mixture prepared according to the protocol was measured at 1-minute intervals using a 37° C. isothermal fluorescence detector, and the results are shown in FIG. 4.

As shown in FIG. 4, it was confirmed that the detection sensitivity was extremely high, at the level of 1 copy.

### Example 4: Evaluation of Detection Specificity

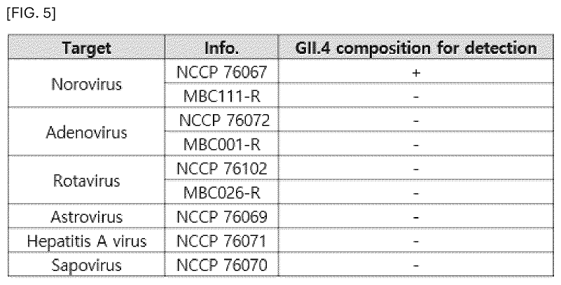

Detection experiments were conducted on various food-related viruses using the same method as in Example 3. As a result, as shown in FIG. 5, viruses other than norovirus were not detected, and furthermore, Norovirus GII.3 was also not detected, confirming extremely high specificity.

### INDUSTRIAL APPLICABILITY

The RNA provided by the present invention enables the rapid detection of norovirus GII.4 in a sample with very high sensitivity and specificity, thereby having very high industrial applicability.

1. (canceled)

2. A composition for detecting norovirus GII.4 comprising:
a guide RNA specific for norovirus GII.4 comprising the nucleotide sequence of SEQ ID NO: 1;
a forward primer comprising the nucleotide sequence of SEQ ID NO: 5;
a reverse primer comprising the nucleotide sequence of SEQ ID NO. 7; and
a CRISPR-associated protein.

3. (canceled)

4. (canceled)

5. The composition according to claim 2, wherein the CRISPR-associated protein is selected from the group consisting of Cas12, Cas13, and Cas14.

6. The composition according to claim 2, wherein the composition further comprises a reporter gene.

7. The composition according to claim 6, wherein the reporter gene is labeled with a detectable label.

8. A kit for detecting norovirus GII.4 comprising the composition of claim 2.

9. A method for detecting norovirus GII.4, comprising:
(a) amplifying nucleic acid obtained from a sample;
(b) adding the composition according to claim 2, and a reporter gene to the amplified product and reacting; and
(c) detecting a signal of the reporter gene.

10. The method according to claim 9, wherein the sample is a food or biological sample.

11. A composition for diagnosing food poisoning comprising the composition of claim 2.

12. (canceled)

13. (canceled)


SEQUENCE LISTING (8 sequences)

SEQ ID NO: 1 - RNA, 24 nt, synthetic construct
  source 1..24: mol_type = other RNA
tacaagcccctggtggagagttca

SEQ ID NO: 2 - DNA, 32 bp, synthetic construct
  source 1..32: mol_type = other DNA
ctcgtmccagaggtcaacaatgaggttawrks

SEQ ID NO: 3 - DNA, 32 bp, synthetic construct
  source 1..32: mol_type = other DNA
agcgggccaacaaaatgtaattgacccctgga

SEQ ID NO: 4 - DNA, 32 bp, synthetic construct
  source 1..32: mol_type = other DNA
tgccgctattgcggcrcctgtagcgggccaac

SEQ ID NO: 5 - DNA, 32 bp, synthetic construct
  source 1..32: mol_type = other DNA
attgcggcrcctgtagcgggccaacaaaatgt

SEQ ID NO: 6 - DNA, 32 bp, synthetic construct
  source 1..32: mol_type = other DNA
tgtyggtgccgctattgcggcrcctgtagcgg

SEQ ID NO: 7 - DNA, 32 bp, synthetic construct
  source 1..32: mol_type = other DNA
gattragytcagggcctaygggcgcgctccat

SEQ ID NO: 8 - DNA, 24 bp, synthetic construct
  source 1..24: mol_type = other DNA
tacaagcccctggtggagagttca